(19) United States
(12) Patent Application Publication
(10) Pub. No.: US 2013/0303783 A1
(43) Pub. Date: Nov. 14, 2013

(54) DEVICE AND PROCESS FOR CONTINUOUS PHOSGENATION

(71) Applicants: Raimondo Pilia, Cagnes-sur-Mer (FR); Alexandre Trani, Carros (FR); Sabrina Marie, Revest-les-Roches (FR); Fabrice De Panthou, Paris (FR)

(72) Inventors: Raimondo Pilia, Cagnes-sur-Mer (FR); Alexandre Trani, Carros (FR); Sabrina Marie, Revest-les-Roches (FR); Fabrice De Panthou, Paris (FR)

(73) Assignee: AET GROUP, Gilette (AT)

(21) Appl. No.: 13/816,785

(22) PCT Filed: Sep. 28, 2011
(86) PCT No.: PCT/FR2011/000530

§ 371 Date: Jun. 7, 2013

(30) Foreign Application Priority Data
Sep. 30, 2010 (FR) 10/03879

(51) Int. Cl. B01J 19/00; C07C 249/02; C07C 68/02; C07D 233/60

(52) U.S. Cl. CPC B01J 19/0046; C07D 233/60; C07C 249/02; C07C 68/02
USPC 548/313.7; 562/802; 558/280; 422/605

(57) ABSTRACT

A continuous process for generation of phosgene from CO and Cl₂ and consumption of the phosgene thus generated in a liquid-phase reaction so as to form organic products P. The process is implemented in two successive reactors R1 and R2, the first reactor R1 being a reactor for catalytic synthesis of phosgene from CO and Cl2 gas, and the second reactor R2 being a piston reactor, the second reactor R2 being equipped with a mechanical axial agitation device.

### CROSS-REFERENCE TO RELATED APPLICATIONS

The present application is a National Stage Application of PCT International Application No. PCT/EP2011/000530 (filed on Sep. 28, 2011), under 35 U.S.C. §371, which claims priority to French patent application Ser. No. 10/03879 (filed on Sep. 30, 2010), which are each hereby incorporated by reference in their respective entireties.

### BACKGROUND

Phosgene (methanoyl dichloride, CAS no. 75-44-5) is a molecule that is very useful in organic synthesis. It may be synthesized very easily by causing chlorine (Cl₂) and carbon monoxide (CO) to react in the presence of a fixed bed catalyst; this reaction is exothermic. Methods for implementing this process are described in numerous very old patents, for example FR 502,672 and 502,673 (L'Air Liquide), filed in 1915 and granted in 1920, U.S. Pat. No. 2,457,493 (D. Bradner), as well as in patent documents FR 2 109 186 (French Government), FR 2 297 190, FR 2 297 191 and FR 2 345 394 (Tolochimie), EP 0 134 506, U.S. Pat. No. 4,764,308 and U.S. Pat. No. 6,930,202 (Bayer AG), MX 164 753 (Antonio Laville Conde), EP 0 796 819 B1 (Idemitsu Petrochemicals Co.), US 2005/0118088 and EP 1 485 195 (BASF AG), US 2005/0025693 (General Electric Co.), and US 2006/0047170 (Bayer Material Science LLC).

The nature of the catalyst was explored in numerous studies; see, in particular FR 2 383 883 (Ube Industries), EP 0 881 986, U.S. Pat. No. 6,022,993, U.S. Pat. No. 6,054,107, U.S. Pat. No. 6,054,612 and EP 0 912 443 (E.I. Du Pont de Nemours), US 2002/0065432 (Eckert et al.).

As a chlorine source, it is also possible to use NOCl (see U.S. Pat. No. 1,746,506 (Du Pont Ammonia Corp.)), CCl4 (see FR 2 100 343 (Farbwerke Hoechst)), HCl (FR 2,144,960 and U.S. Pat. No. 3,996,273 (Societe Rhône-Progil)), or C2Cl4 (U.S. Pat. No. 5,672,747 (J. Stauffer)). Some of these products are more expensive and/or more hazardous than chlorine, and no easier to handle.

The recycling of effluents in the synthesis of phosgene has always been a major concern; see U.S. Pat. No. 4,231,959 (Stauffer Chemical Corp.) and US 2009/0143619 (Bayer Materials Science AG). The synthesis of phosgene has even been envisaged as a means for selectively reusing the chlorine contained in industrial effluents (see U.S. Pat. No. 4,346,047 (The Lummus Company)).

In organic synthesis, phosgene is a fairly polyvalent molecule because it enables four different families of reactions to be carried out:

(i) carbonylation, which leads to isocyanates and urea,

(ii) chloroformylation, which leads to chloroformates, from which carbamates, carbonates and diaryl ketones can be obtained,

(iii) chlorination, leading in particular to alkyl or aryl chlorides,

(iv) dehydrogenation, leading to cyanides, isocyanides and carbodiimides.

As an example, it is used industrially, for example, for the synthesis of dialkyl carbonates from alcohols. Certain 1,2- and 1,3-diols form, with phosgene, cyclic carbonate esters. Thus, 1,2-ethane-diol (also called ethylene glycol) forms ethylene carbonate (formate); this reaction involves the formation of an intermediate chlorocarbonate ester. When, in the diols, the hydroxy groups are separated by more than 3 carbon atoms, the cyclization is slow, and the reaction generally leads to a polymer.

The disadvantage of phosgene is its very high toxicity; therefore, it is subject to very stringent regulations for storage and transport in particular. Moreover, phosgene is well known to the general public because it was used as a chemical warfare agent in the World War I. It is known that phosgene can be destroyed by contact with an alkaline solution; thus U.S. Pat. No. 4,493,818 (Dow Chemical Co.) describes a process for destroying phosgene by contact with an aqueous alkaline solution containing a tertiary amine.

Phosgene is used on a large scale in the industrial preparation of polycarbonate-type polymers, as well as isocyanates, which are raw materials in the synthesis of polyurethane polymers. As an example, U.S. Pat. No. 5,986,037 (Mitsubishi Chemical Corp.) describes the production of a solution of polycarbonate oligomers from bisphenol and phosgene. US 2001/0041806 A1 and EP 1 112 997 B1 (M. Miyamoto and N. Hyoudou) describe the production of diaryl carbonates by condensation of an aromatic monohydroxy compound with phosgene; in this process, the chlorine formed by this reaction is reused in the production of phosgene. These processes for use of phosgene are generally continuous processes.

At this industrial scale, phosgene has such considerable cost advantages that it is accepted to manage the risks inherent to this gas. To minimize the risk in storage, it can be stored and used in the form of a solution. Thus, U.S. Pat. No. 3,226,410 (FMC Corporation) describes a continuous reactor for producing aromatic isocyanates from amines and a phosgene solution in an organic solvent (monochlorobenzene). Similarly, U.S. Pat. No. 3,321,283 (Mobay Chemical Co.) describes a large tubular reactor for phosgenation of amines using a phosgene solution in ortho-dichlorobenzene.

However, the minimization of the risk involves the direct synthesis of phosgene, in order to avoid its transport, and the reuse of all residual gases. The latter is also required for economic reasons. Thus, US 2006/0123842 and US 2007/0012577 (BASF) as well as US 2007/0269365 (Bayer Material Science AG) describe a process for reusing HCl generated in the synthesis of isocyanates from amines and phosgene, with this reuse involving the oxidation of HCl into Cl2, with the latter being reused for the synthesis of phosgene. In the same context US 2007/0276158, US 2007/0276154 and US 2009/0054684 (Bayer Material Science AG) describe a process for reusing excess CO from the synthesis of phosgene, mixed with HCl generated during the synthesis of isocyanates, in the synthesis of phosgene. WO 2009/143971 (Bayer Technology Services GmbH) describes a phosgene synthesis reactor with multiple catalytic zones enabling a quasi-quantitative reaction, which reduces the quantity of effluents to be treated. U.S. Pat. No. 5,925,783 (Bayer AG) describes a process for producing isocyanates from amines and phosgene, with the phosgene being introduced into the reactor in a solution with an isocyanate, which may be the targeted isocyanate. US 2009/0209784 (Bayer Material Science) describes an improvement in the separation and recycling of residual phosgene and the HCl from the amine phosgenation reaction products.

The document “Production industrielle avec le phosgene”, Chimia (1998) 52(12), 698-701, [“Industrial production with phosgene”], describes a plant for dynamic production and use of phosgene gas used by Novartis Crop Protection (ex. Ciba Geigy). In this plant, all of the parts capable of containing phosgene are in a double envelope continuously swept with air, which is continuously analyzed and ends up in two consecutive NaOH 10% washing towers in order to destroy the phosgene in the event of a leakage. All parts of the plant are controlled and regulated by computer. The article “Safe phosgene production on demand” (U. Osterwalder, Ciba-Geigy Werke Schweizerhalle A.-G., Bericht über das Internationale Kolloquium über die Verhütung von Arbeitsunfullen and Berufskrankheiten in der Chemischen Industrie (1985), 10 (785-800)), describes the development of this same plant, and mentions in particular the development of a laboratory apparatus capable of producing phosgene quantities of approximately 40-600 kg/h on demand.

Numerous practical aspects must be taken into account in the design of phosgenation reactors. The phosgenation of amines is a highly exothermic reaction that occurs even at low temperature. The way in which an intimate mixture of the starting products (amine and phosgene) is obtained as quickly as possible thus becomes a critical factor capable of limiting the yield and leading to solid secondary byproducts. U.S. Pat. No. 7,547,801 (Bayer Material Science) describes a three-step process in which the different intermediate reactions of the reaction are spatially separated. FR 2 940 283 {Perstop Tolonates France) describes a process in which a phosgene jet is injected at very high rate (around 10 to 100 m/s) in a turbulence zone specifically provided in a piston reactor at high-pressure (5 to 100 bar, preferably 20 to 70 bar) and high temperature (100° C. to 300° C.) in order to continuously convert amines into isocyanates; this process, owing to the turbulent mixture of reagents and owing to the high pressure of the phosgene jet, makes it possible to obtain a very short phosgenation reaction time (less than 200 ms). However, it involves the use of an excess of phosgene at high pressure, two major disadvantages.

U.S. Pat. No. 5,931,579 (Bayer AG) describes a phosgene injection nozzle system, which prevents clogging by solid byproducts, thus minimizing the need to open and disassemble the reactor, a cumbersome procedure that must be preceded by a complete purge and that requires very strict safety measures. Moreover, it appeared that the use of tubular reactors for gas-phase phosgenation of amines presents numerous practical problems. It has therefore been sought to use other types of reactors; thus, U.S. Pat. No. 7,084,297 (BASF AG) describes a process for producing isocyanates by gas-phase phosgenation of primary amines in a plate reactor.

In the production of fine chemicals, in particular for the production of active principles for drugs, fragrances, phytosanitary products and the like, there are relatively few examples of the direct use of phosgene. More often, phosgene is generated from diphosgene or triphosgene; the first is a liquid under normal conditions, the second forms crystals. The patent U.S. Pat. No. 6,399,822 (Dr. Eckert GmbH) describes a catalytic process for generating pure phosgene from diphosgene or triphosgene; a device implementing this patent is sold by the Sigma Aldrich company and is described in the journal ChemFiles, vol. 7, no. 2 (2007), pages 4 and 14. It is suitable for very small-scale applications, in particular for the derivatization of amino acids and peptides. Similarly, there are publications relating to microreactors producing minimal amounts of phosgene. These reactors, of submillimetric dimensions, are produced by techniques based on microelectronics, in particular photolithography and ion etching. Thus, the final technical report no. AFRL-IF-RS-TR-2002-295 “Micro-Fluid Chemical Reactor Systems: Development, Scale-Up and Demonstration” of the Massachusetts Institute of Technology mentions a microreactor for the direct synthesis of very small quantities of phosgene with a standard Cl2 flow rate of 4 cm3/min. The article “Microfabricated packed-bed reactor for phosgene synthesis” by S. K. Ajmera et al., published in the AlChE Journal, vol. 47(7), p. 1639-1647 (2010), describes a microreactor with a standard 2 CO+Cl2 flow rate of 4.5 cm3/min. US 2004/0156762 also mentions the use of a microreactor for the direct synthesis of phosgene.

The article “Challenging chemistries at Saltigo: A competitive advantage in custom manufacturing” by Ann Gidner, published in the journal Chemistry Today, vol. 24, n. 5, page 50-52 (2006), expresses the desire to develop alternatives to the direct use of phosgene in the production of fine chemicals.

However, diphosgene and triphosgene are significantly more expensive than phosgene. But in the production of fine chemicals, for example, for the synthesis of pharmaceutical active principles, aromas or fragrances, the raw material cost factor of is not always a very important argument. For this reason, and for safety reasons, there are, in these industries, few processes that involve the direct use of phosgene. In general, alternative processes that avoid the use of certain products such as phosgene continue to be sought, as demonstrated, for example, by the article “Products and processes for a sustainable chemistry: a review of achievements of prospects” by J. Jenck et al., published in the journal Green Chemistry 6, p. 544-556 (2004). In some cases, phosgene substitutes are also used, such as the Vilsmeier reagent or carbonyldiimidazole.

The present invention goes against this tendency and seeks to propose a device and a process for phosgenation that are suitable for the production of fine chemicals, with the process involving the injection of phosgene into a liquid-phase reactor, optionally at high pressure, capable of producing and consuming significant but limited quantities of phosgene, i.e. approximately 1 to 50 kg/h, and in particular 1 to 5 kg/h, and capable of being implemented under impeccable safety conditions, in particular without any phosgene storage.

### SUMMARY

The invention first relates to a continuous process for generation of phosgene from CO and Cl₂ and consumption of the phosgene thus generated in a liquid-phase reaction so as to form organic products P, wherein said process is implemented in two successive reactors R1 and R2,

the first reactor R1 being a reactor for catalytic synthesis of phosgene from CO and Cl2 gas,

the second reactor R2 being a piston reactor, preferably cylindrical, and said second reactor R2 being equipped with mechanical axial agitation means,

and in which process:
preferably stoichiometric quantities of CO and Cl2 are introduced in said reactor R1 in order to form a gas mixture,
said gas mixture is passed over a suitable catalyst to form phosgene,
said gas mixture comprising phosgene is passed through said second reactor R2,
at least one liquid phase comprising an organic compound E is continuously introduced, preferably at one end of said second reactor R2,
said liquid phase is subjected, preferably at a temperature of between −25° C. and 200° C. and under mechanical axial agitation, to the influence of a phosgene pressure of between 1 and 10 bar, and preferably between 1 and 5 bar, optionally in the presence of a liquid-phase dispersed catalyst, for a passage time t of between 1 second and 10 minutes, and preferably between 10 seconds and 6 minutes, and more preferably between 40 seconds and 3 minutes,
the liquid phase is removed from said reactor,

and in which, preferably, the increase in temperature ΔT of the liquid between the inlet and the outlet of the reactor is such that the ratio ΔT/ΔTad (where ΔTad represents the adiabatic temperature increase) is between 0.02 and 0.6 when the ratio between the characteristic heat transfer time ttherm and the characteristic material transfer time tmat is between 1 and 50.

The invention also relates to a device for implementing the process described above, comprising a first reactor R1 located upstream of a second reactor R2, said first reactor R1 being located in a first closed compartment C1, and said second reactor R2 being located in a second closed compartment C2, compartments C1 and C2 being separated by a wall, wherein

said first reactor R1 is a phosgene generating reactor comprising:
a tank and an inlet for carbon monoxide gas with flow regulator thereof,
a tank and an inlet for chlorine gas with a flow regulator thereof,
a reaction chamber, preferably tubular, comprising a fixed bed catalyst enabling the carbon monoxide—chlorine gas mixture to be converted into phosgene,

said second reactor R2 is a tubular phosgenation reactor, comprising:
at the upstream end, an inlet for the gaseous reaction product coming from said first reactor R1,
at the upstream end, an inlet for a liquid phase comprising said organic compound,
at the downstream end, an outlet for said liquid phase,

wherein said first reactor R1 and said second reactor R2 are connected to one another by a tube for transfer of the reaction product comprising the phosgene generated in said first reactor R1 to said second reactor R2, with said tube passing through said wall between said first compartment C1 and said second compartment C2.

In addition, in the device according to the invention:
said second reactor R2 is a piston reactor equipped with mechanical axial agitation means,
said second reactor R2 operates at a pressure of between 1 and 10 bar and preferably between 1 and 5 bar.

Advantageously, said transfer tube includes means for cooling the phosgene, preferably means enabling a temperature of approximately 30° C. to be obtained.

The invention also relates to the use of a device according to the invention for phosgenation of an organic compound.

### DESCRIPTION

The invention relates to a continuous process for phosgenation of an organic compound, wherein said process is implemented in a continuous reactor of the piston reactor type (also called a piston flow reactor), with a length L and a volume V, in which the chemical species enter at one end and move throughout the reactor while gradually being converted.

The continuous process according to the invention can be described as comprising a plurality of steps.

In a first step, phosgene is generated in the first reactor R1, from CO and Cl2. A liquid phase comprising said organic compound is introduced into the reactor R2, continuously, preferably at one end of said reactor.

Then said liquid phase is subjected, at a temperature of between −25° C. and +200° C. and under mechanical axial agitation, to the influence of a phosgene pressure of between 1 and 10 bar (preferably between 1 and 5 bar) for a passage time t of between 1 second and 10 minutes (preferably 10 seconds and 6 minutes, and even more preferably 40 seconds to 3 minutes).

When the liquid phase comprising the product P reaches the downstream end of the reactor R2, it is removed at said downstream end. The liquid phase may contain gas such as excess CO, CO2 or HCl generated during the reaction. It may also contain solids (reaction product or byproduct).

The phosgene used for the phosgenation is produced in a reactor R1 by reacting chlorine with carbon monoxide, in the presence of a catalyst, generally activated carbon. Other catalysts, such as metal halogenides and silicon carbide can also be used. As the phosgene synthesis reaction is highly exothermic, it is necessary to cool the reactor R1. The reaction can advantageously be produced at a temperature of around 150° C., and more generally between 50° C. and 200° C. At the outlet of the reactor R1, the phosgene is cooled so as to obtain a temperature of approximately 30° C. at a pressure of between 1 and 10 bar, and preferably between 1 and 5 bar. It is at this pressure that the phosgene is injected into the upstream end of the reactor R2, which is a piston reactor. The phosgene pressure at the outlet of the reactor R1 is generally sufficient to supply the reactor R2. Consequently, it is not necessary to use an intermediate pump between R1 and R2 to increase the pressure of the phosgene entering the reactor R2. This makes the process according to the invention safer by reducing the risk of a phosgene leakage. This is one of the advantages of the process according to the invention. However, for some reactions, it may be necessary to use an intermediate pump, for example when highly volatile solvents are used. In this case, it is possible to implement the process according to the invention with a pressure in the reactor R2 of up to around 200 bar.

In the process according to the invention, the flow rate of the CO and Cl2 gases entering the first reactor R1 is preferably controlled by the phosgene consumption in the phosgenation reactor R2, given that an approximately stoichiometric quantity of phosgene is very preferably introduced into the reactor R2; it is, however, preferable to use a slight excess of CO, in order to avoid obtaining a phosgene that contains chlorine. This control of the gas flow rate at the inlet of the reactor R1 by the phosgene consumption at the outlet of the reactor R2 can be performed with mass flowmeters at the inlet of each of the CO and Cl2 gaz. Advantageously, these flowmeters do not allow gas to pass unless the reactor R2 is operational.

The phosgene gas generated in the reactor R1 is then immediately transferred to the reactor R2.

Most of the phosgenation reactions carried out in the reactor R2 require the use of phosgene of high purity, greater than or equal to 99%.

The reactor R2 used for the phosgenation preferably has a cylindrical shape. It must be equipped with axial agitation means, preferably mechanical axial agitation means. By axial agitation means, we mean any device that performs the agitation of the reaction mixture over the entire length, or a significant portion thereof, by means having an axis parallel to the axis of the reactor. These axial agitation means facilitate, on the one hand, the reaction, by mixing the chemical species introduced with the catalyst, which is in dispersed form in a liquid phase, and, on the other hand, facilitate the heat transfer.

The piston reactor has temperature and concentration profiles that may vary along its axis. Such a reactor can be modeled as a series of basic reactors arranged in series along an axis and each having a length ΔL and a volume ΔV. Under the process conditions of this reactor, the composition of the feed and the total volumetric flow rate F are uniform and constant, and the residence time:

τ=V/F (Equation 1)

is constant for all of the molecules entering the reactor. This type of reactor is known, and a person skilled in the art also knows that if a highly exothermic reaction is produced in a piston reactor, the radial heat transfer may become limiting. For phosgenation reactions, which may be highly exothermic, the control of heat transfers is therefore critical.

The process according to the invention involves a chemical reaction of the following type:

A(liquid)+νCOCl2(gas)−>νp Product (Equation 2),

where ν is the stoichiometric coefficient of phosgene and νp is the stoichiometric coefficient of the product. According to the invention, the organic compound that undergoes the phosgenation is in the form of a pure liquid or is a liquid diluted in a liquid solvent, or is in the form of a solid diluted in a liquid solvent.

In general, the performances of reactors are given by two characteristic quantities, which respectively describe the heat transfer and the material transfer. These characteristic transfer times are defined below by simplified equations (the hydrodynamic model being the same, whether the reactor is a piston reactor or a fully agitated reactor, insofar as the phosgenation reaction is limited by the material transfer):
characteristic heat transfer time:

$${t}_{therm} = \frac{{pC}_{p} {V}_{liq}}{KS} \qquad ( Equation 3 )$$

characteristic material transfer time:

tₘₐₜ=1/(k_{L}a)

In these equations, the following parameters are used: the density of the liquid p,
the calorific capacity of the liquid Cp; the overall transfer coefficient K, defined below; heat exchange surface S (constant for a given reactor, because it is fixed by design); the product between the gas-liquid material transfer coefficient on the liquid side kL and the specific interfacial area, a, defined below.

The shorter the characteristic transfer time is, the better the system performs and rapidly transfers the heat and the material (respectively).

Here, we briefly describe the determination of the coefficient K well known to a person skilled in the art.

The overall transfer coefficient K (also called the overall exchange coefficient) is defined by the equation:

φ=KSΔTml (Equation 5),

where S is the exchange surface (in this case, for a cylindrical reactor, S=πDL,

where D is the interior diameter of the reactor and L is the interior length of the portion of the tube of the reactor in which the gas comes into contact with the liquid), ΔTml is the mean logarithmic temperature difference:

ΔTml={[(T(coolant)outlet−T(process)inlet]−[(T(coolant)inlet−T(process)outlet]}/In{[(T(coolant)outlet−T(process)inlet]/[(T(coolant)inlet−T(process)outlet]}

and φ is the power (in Watts, reference temperature 25° C.) gained by the heat flux on the process side. For a given reaction, these parameters are dependent on the geometry of the reactor and the flow rate; they can be determined easily.

The coefficient kLa, also well known to a person skilled in the art, can be determined experimentally by a procedure that, so as not to unnecessarily complicate the description of the invention, is described below as “Example 1.”

In an advantageous embodiment of the invention, a continuous piston-type reactor having the following characteristics is used:
material transfer: 0.1 s-1<kLA<0.3 s-1, that is, 3 s<tmat<10 s
heat transfer: K=300 to 1000 W/m2/° C. (preferably 300 to 700 W/m2/° C., and even more preferably about 550 W/m2/° C.) (here, the partial transfer coefficient of liquid with metal is considered).

In a typical embodiment, the characteristic transfer time

$${t}_{therm} = \frac{{pC}_{p} {V}_{liq}}{KS}$$

is approximately 25 seconds (with p=1050 kg/m3, Cp=200 J/kg/° C.).

In this advantageous embodiment, the ratio of characteristic times is therefore:

2<(ttherm/tmat)<8

In the process according to the invention, the increase in temperature of the liquid ΔT between the inlet and the outlet of the reactor is such that:

$$\frac{Δ T}{Δ {T}_{ad}} = \frac{{t}_{therm}}{( {t}_{therm} + {τ}_{liq} )} {X}_{A} , \qquad ( Equation 6 )$$

where ΔTad is the adiabatic temperature increase.

$$Δ {T}_{ad} = \frac{( - {Δ}_{r} H ) {C}_{A 0}}{{pC}_{p}} , \qquad ( Equation 7 )$$

where ΔrH is the enthalpy of the reaction and XA is the stoichiometric coefficient of compound A.

For the case of a total conversion of A (i.e. XA=1), equation (6) can be rewritten as:

$$\frac{Δ T}{( Δ {T}_{ad} )} = \frac{M}{( {t}_{mat} / {t}_{therm} + 1 )} , \qquad ( Equation 8 )$$

where M designates the stoichiometric ratio:

$$M = \frac{P}{{vHeC}_{A 0}} \qquad ( Equation 9 )$$

in which P signifies the working pressure, He signifies the Henry coefficient, and CA0 signifies the concentration of the liquid at the inlet of the reactor.

The choice of the process conditions of the process according to the invention involves three quantities:
the adiabatic temperature increase in a non-diluted medium:

$${( Δ {T}_{ad} )}_{pure} = \frac{( - {Δ}_{r} H ) {( {C}_{A 0} )}_{pure}}{{pC}_{p}} \qquad ( Equation 10 )$$

the stoichiometric ratio calculated on the concentration of the pure reagents:

$${M}_{pure} = \frac{P}{{vHe ( {C}_{A 0} )}_{pure}} \qquad ( Equation 11 )$$

the dilution factor F defined by

$${( {C}_{A 0} )}_{working} = \frac{{( {C}_{A 0} )}_{pure}}{F} \qquad ( Equation 12 )$$

The inventors discovered that a particular operating condition of a piston reactor enables the stated problem to be solved. This operating condition is explained here in the case of a reaction with a stoichiometric coefficient vp=1, as is the case, for example, for the chloroformate reaction. This rate concerns both a flow rate and an agitation rate.

Indeed, according to the invention, the reaction is conducted so that the increase in temperature ΔT of the liquid between the inlet and the outlet of the reactor is such that the ratio ΔT/ΔTad (where ΔTad represents the adiabatic temperature increase) is between 0.02 and 0.6 when the ratio between the characteristic heat transfer time ttherm and the characteristic material transfer time tmat is between 1.5 and 50.

This process can be used without a solvent, i.e. said organic compound constitutes the liquid phase introduced into the reactor. However, it is possible to use a solvent if necessary, and in particular if the phosgenation product or byproduct is a solid (formation of salts). In a preferred embodiment, the ratio ΔT/ΔTad is between 0.02 and 0.2 when ttherm/tmat is between 1.5 and 12. In an even more preferable embodiment, ΔT/ΔTad is between 0.03 and 0.15 when ttherm/tmat is between 2 and 8.

To keep the internal temperature of the reactor constant, the heating or cooling power of the reaction chamber R2 is adjusted.

Advantageously, the phosgenation step of the process according to the invention is implemented in a cylindrical tubular piston reactor R2, with an interior diameter of between 20 mm and 100 mm. Above 100 mm, the productivity of the reactor decreases because, in order for the exchange surface to remain large, it is necessary to reduce the flow rate. Below 20 mm, the surface/volume ratio is very high, but the flow rate is insufficient for industrial production. Preferably, the interior diameter of the piston reactor is between 30 mm and 75 mm, and even more preferably between 40 mm and 60 mm. The length of the reaction chamber of the reactor is between 10 cm and 100 cm. Below 10 cm, the residence time is very short. Above 100 cm, the machining of the tubular reactor becomes difficult, and the agitation of the reaction mixture is difficult to achieve. A preferred length is between 20 cm and 80 cm.

The reactor R2 must be equipped with axial agitation means, preferably mechanical axial agitation means. Different means can be used to this end, such as a series of mixers, an endless screw, or a stirrer, but these mechanical agitation means must not disrupt the “piston” character of the reactor, as defined by equation (1).

The phosgenation process according to the invention does not need a catalyst. However, if a catalyst is used, it is possible to use at least one dispersed catalyst, such as a powder in suspension. Advantageously, this powder consists of a support (such as alumina, silica or activated carbon) on which a suitable metallic element has previously been deposited.

The process according to the invention has numerous advantages. For certain reactions, it is possible to find process conditions in which the reaction yield is greater than or equal to 99%. This high yield makes it possible to avoid additional purification steps that are often necessary in the synthesis of fine chemicals when the yield is below 95%. However, not all reactions enable such a high yield to be obtained. If the phosgenation product is not a solid, it is possible to work in some cases without a solvent. The reactor makes it possible to consume around 1 to 50 kg/h of phosgene, and preferably between 1 and 5 kg/h. Moreover, the investment cost of a reactor capable of implementing the process according to the invention is lower than that of a batch-type reactor, and the manpower requirements are reduced.

Advantageously, said product P obtained by the phosgenation of an organic compound is selected from the group consisting of carbonyl chlorides, chloroformates, carbamates, isocyanates, carbamoyl chlorides, urea and derivatives thereof. It is also possible to prepare acid chlorides; for this, a heterogeneous catalyst (suspension) is advantageously used, but the inventors observed that the chlorination often requires an excessively long reaction time. In this case, the reaction does not have a good yield and the outlet of the residual phosgene reactor must be managed; this embodiment is not therefore a preferred embodiment.

Chloroformates:

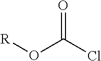

Acid chlorides:

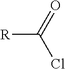

Carbamates:

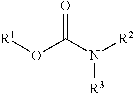

Isocyanates: R—N═C═O

Carbamoyl chlorides:

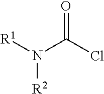

Ureas:

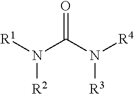

Carbonates:

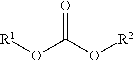

In the formulas above, R, R1, R2, R3 and R4 can represent a substituent such as an aliphatic group, linear or branched, or an aralkyl group, an alkenyl group, a cycloalkyl group, or a cycloalkenyl group, all of these groups advantageously containing between 1 to 36 carbon atoms.

The groups R—N—, R1(R2)-N—, R2(R3)-N— and R3(R4)-N— can represent a pyrrole, a 2-pyrroline, a 3-pyrroline, a pyrrolidine, an imidazole, a 2-imidalozine, an imidazolidine, a pyrazole, a 2-pyrazoline, a pyrazolidine, a 1,2,3-triazole, a piperidine, a piperazine, an indole, an isoindole, an indolione, a 1H-indazole, a benzimidazole, a purine, a carbazole, a phenothiazine, a phenoxazine, an aziridine, an azetidine, a morpholine, a thiomorpholine, substituted or not by saturated or unsaturated groups, mono- or polysubstituted, or a derivative of one or these heterocyclic systems that keeps its cyclic character intact. Below are some examples of more complex derivatives of these heterocyclic molecules:

Indole derivatives: tryptophan, tryptamine, reserpine.

Imidazole derivatives: histidine

Purine derivatives: adenine, guanine, theobromine.

The R—N—, R1(R2)-N—, R2(R3)-N— and R3(R4)-N— groups can represent a heterocyclic compound containing at least one nitrogen atom in the ring that has a covalent bond outside the ring. In this context, it is possible to cite the derivatives of pyridazine, pyrimidine and pyrazine, (for example cytosine, thymine, uracil), quinazoline, quinoxaline, quinazoline, phenazine, and 1,2,3-oxadiazole.

Below are some examples of chemical reactions that may be produced in the reactor R2 by using the process according to the invention.

1) Preparation of 1,1′-carbonyldiimidazole (CAS no. 530-62-1) from imidazole:

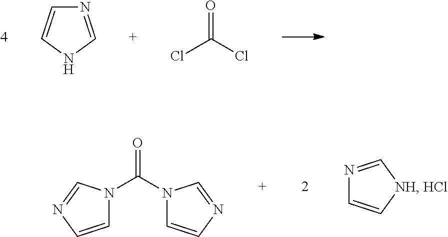

2) Preparation of (chloromethylene)dimethyliminium chloride (CAS no. 3724-43-4) (“Vilsmeier reagent”):

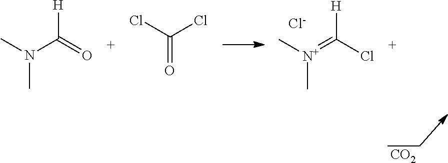

3) Preparation of 1,1′-carbonyl-di(1,2,4-triazole)) (CAS no. 41864-22-6):

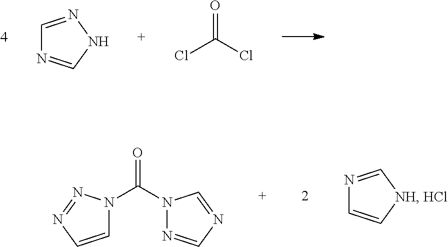

4) Preparation of 5-chlorovaleroyl chloride (CAS no. 1575-61-7)

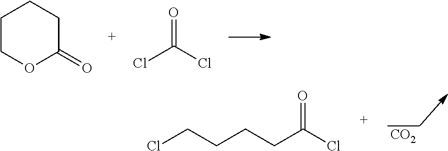

5) Preparation of phthaloyl chloride (CAS no. 88-95-9):

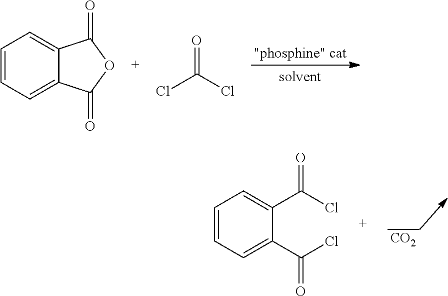

6) Preparation of 2-chloro benzimidazole: This example shows how the process according to the invention can be used elegantly in multi-step syntheses:

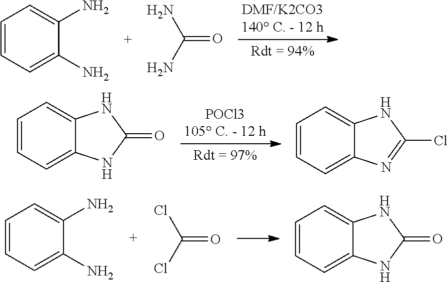

7) Preparation of diphenyl carbonate (CAS no. 102-09-0):

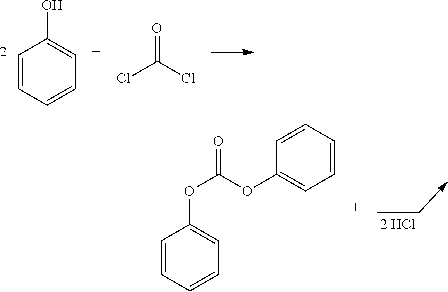

The invention also relates to a device for implementing the process described above. The device comprises a first reactor R1 located upstream of a second reactor R2, said first reactor R1 being located in a first closed compartment C1, and said second reactor R2 being located in a second closed compartment C2, with compartments C1 and C2 being separated by a wall.

The first reactor R1 is a catalytic phosgene generating reactor comprising a tank and a carbon monoxide gas inlet with its flow regulator, a tank and a chlorine gas inlet with its flow regulator, a reaction chamber, preferably tubular, comprising a fixed bed catalyst, enabling the carbon monoxide—chlorine gas mixture to be converted into phosgene.

The second reactor R2 is a tubular reactor, comprising, at its upstream end, an inlet for the gaseous reaction product coming from said first reactor R1, at its upstream end, a liquid phase inlet comprising the organic compound intended to undergo phosgenation, and, at its downstream end, an outlet for said liquid phase, including the phosgenation product P. The first reactor R1 and the second reactor R2 are connected to one another by a tube for transfer of the reaction product generated in said first reactor R1 and comprising phosgene, to said second reactor R2, said tube passing through the wall separating said first compartment C1 and said second compartment C2. This transfer tube can be fairly short, approximately 10 to 30 cm. Advantageously, it comprises at least one flexible zone enabling mechanical stresses to be accommodated; this helps to minimize the risk of breakage of the transfer tube or of its attachment to the ends of said reactors R1 and R2.

Furthermore, the second reactor R2 is a piston reactor equipped with mechanical axial agitation means. The second reactor R2 operates at a pressure of between 1 and 200 bar, and preferably between 1 and 5 bar.

In an advantageous embodiment, said first compartment C1 and said second compartment C2 of the device according to the invention are under a negative pressure with inert gas sweeping (for example, nitrogen), with their gas content being continuously suctioned from above and from below, and sent through a wet scrubber, capable of destroying the phosgene and the chlorine gas that said gas content may contain. The separation of said first and second compartments C1, C2 by a wall does not need to be perfectly tight, but the two compartments must be sealed from the environment, with the only gas outlet passing through the scrubber.

The device according to the invention also comprises control means such as a suitable data processing system. Preferably, the carbon monoxide and chlorine flow regulators of the reactor R1 are controlled by the phosgene demand of the phosgenation reaction in reactor R2. In one embodiment, the phosgene demand is calculated and the CO and Cl2 gas flow rate is consequently adjusted, making sure to maintain a slight excess of CO.

The process according to the invention has numerous advantages. It eliminates the handling of phosgene because the amount of free phosgene in the device is very low. It eliminates the handling of residual gases containing a significant amount of phosgene. It is very flexible and makes it possible to produce a very large number of different molecules with very good yields, implementing different reaction mechanisms. In consideration of the size of the reactor R2, the device is very suitable for the synthesis of fine chemicals; the size of the reactor R2 cannot be increased beyond certain limits because the heat transfer and/or the material transfer in the piston reactor then becomes limiting. The reactor R2 is capable of absorbing irregularities in the production of phosgene, as they may appear in particular when the process is started up, before a steady state is obtained.

### EXAMPLES

The invention is illustrated below in examples 1 to 3, which nevertheless do not limit the invention. Example 1 concerns a chemical reaction that does not fall within the scope of the present invention, but that is used here to describe an experimental procedure that can be used to determine the parameter kLa of a piston reactor. Examples 2 and 3 illustrate two typical chemical reactions.

### Example 1

This is an experimental method that can be used to determine the parameter kLa of a reactor.

The product kLa is determined for a given reactor on the basis of a well-known chemical reaction, namely the catalytic hydrogenation of nitrobenzene into aniline (Ph-NO2+3H2->Ph-NH2+2H2O, where Ph designates a phenyl group). This reaction is carried out in the liquid phase without a solvent, with the gas phase consisting of pure hydrogen at an initial pressure of 2 bars. The catalyst consists of carbon powder (equivalent particle diameter of approximately 50 μm) filled to 5% by weight of palladium. The mass concentration of the catalyst is 2.5 g/l and the hydrogenation is performed at ambient temperature. A quartz pressure sensor enables the hydrogen pressure to be measured over time. The reactor has a double envelope; a circulation of thermostatically controlled water inside the double envelope enables the temperature of the reactor to be kept constant. At the start, the non-agitated reactor is kept under nitrogen pressure; it is then purged with hydrogen. At a hydrogen pressure of 2 bars, the agitation is started and the drop in hydrogen pressure is monitored. The reaction is left to continue until the pressure reaches a value of 0.5 atm. Then, the agitation is stopped and the apparatus is repressurized with hydrogen, then after ten minutes the measurement is repeated with a different agitation rate. For each test performed, it is noted that the hydrogen pressure decreases according to an exponential law. Thus, by tracing In PH2/P0=f(t), a straight line is plotted, the slope of which makes it possible to obtain the product a kapp. If the change in this product a kapp with the agitation rate is plotted, asymptotic behavior is observed. For low agitation rates, the apparent conductance increases with the agitation rate; this indicates a limitation in the apparent kinetics for the gas-liquid transfer. For high agitation rates, a plateau is obtained; this indicates that the transfer is limited, either by the chemical kinetics or by the kinetics of the liquid-solid material transfer. The implementation of the curve α kapp=f (agitation rate) then makes it possible to estimate the transfer conductance value kLa.

These are the theoretical bases of this determination of kLa.

Disregarding the accumulation of hydrogen in the liquid phase, the expression of the hydrogen disappearance flux in the closed reactor can be established.

$$\frac{d {n}_{{H}_{3}}}{d t} = - \frac{{V}_{G}}{RT} \frac{d {P}_{{H}_{2}}}{d t} = {ϕ}_{{H}_{2}} {V}_{R} \qquad ( 1 )$$

where φH2 is the specific hydrogen disappearance flux. This flux can be expressed by showing either the reaction rate or the transfer flux:

$${ϕ}_{{H}_{2}} = {r}_{V} α = {( {K}_{{H}_{2}} a )}_{global} ( \frac{{P}_{{H}_{2 }}}{He} - {C}_{{H}_{2}}^{surface} ) \qquad ( 2 )$$

and rv is the volumetric hydrogenation reaction rate, α is the solid retention in the reactor and (KH2a)global is the overall hydrogen transfer conductance from the gas phase to the surface of the catalyst.

As the volume of the catalyst and the nitrobenzene concentration are considered to be invariant during a test, the reaction rate can be expressed as resulting from first-order kinetics with respect to the hydrogen concentration, i.e.:

rᵥ=ηkᵥC_{NB}C_{H}_{₂}^{surface}=kᵥ′C_{H}_{₂}^{surface} (3)

When the gas phase is pure hydrogen, the overall transfer conductance can be expressed as a function of the partial gas-liquid and liquid-solid transfer conductances by:

$$\frac{1}{{[ {K}_{{H}_{2}} a ]}_{global}} = \frac{1}{{K}_{L} {a}_{LG}} + \frac{1}{{k}_{S} {a}_{S} α} \qquad ( 4 )$$

where as is the specific surface of the solid and aLG is the specific gas-liquid surface. By combining the expressions of the chemical kinetics and the physical kinetics, the specific hydrogen disappearance flux in the reactor can be expressed by:

$${ϕ}_{{H}_{2}} = α {k}_{app} \frac{{P}_{{H}_{2}}}{He} \qquad ( 5 )$$

where α kapp is an apparent conductance that integrates the limitations due to the chemical kinetics, but also the limitations due to the physical kinetics.

$$\frac{1}{α {k}_{app}} = \frac{1}{{[ {K}_{{H}_{2}} a ]}_{global}} + \frac{1}{α {k}_{v}^{′}} = \frac{1}{{k}_{L} {a}_{LG}} + \frac{1}{α} [ \frac{1}{{k}_{S} {a}_{S}} + \frac{1}{{k}_{v}^{′}} ] \qquad ( 6 )$$

By injecting (5) into (1), the following is obtained:

$$\frac{{dP}_{{H}_{2 }}}{{P}_{{H}_{2 }}} = - α {k}_{app} \frac{{V}_{R}}{{V}_{G}} \frac{RT}{He} dt \qquad ( 7 )$$

of which the integration leads to:

$$ln \frac{{P}_{{H}_{2 }}}{{P}_{0}} = - α {k}_{app} \frac{{V}_{R}}{{V}_{G}} \frac{RT}{He} ( t - {t}_{0} ) \qquad ( 8 )$$

The interpretation of the change in hydrogen pressure in a closed system thus enables the apparent conductance of the system to be determined. The latter makes it possible to deduce to the gas-liquid transfer conductance value.

### Example 2

In the first reactor R1, a flux of 8.0 g/min (i.e. 0.113 mol/min) of Cl2 and 3.3 g/min (i.e. 0.118 mol/min) of CO (corresponding to a molar ratio CO/Cl2 of around 1.050) were reacted in a reactor according to the invention. The flux leaving the first reactor contains 11.1 g/min of phosgene and 0.2 g/l of excess CO. During the 8 hours of operation in a steady state, 5.4 kg of phosgene are thus produced from 3.8 kg of Cl2 and 1.7 kg of CO, and 0.1 kg of excess CO is recovered.

In the second reactor R2, the flux of 11.1 g/min (i.e. 0.113 mol/min) of phosgene generated in the first reactor is reacted with a solution of 28.1 g/min (i.e. 0.412 mol/min) of imidazole in ethyl acetate introduced in a permanent flux into the second reactor (corresponding to a molar ratio of imidazole/COCl2 of around 4.0). The solution introduced into the reactor has a temperature of 60° C. to 65° C. The reaction itself takes place at a temperature of around 75° C. to 80° C. The solution leaving the reactor is collected in a tank kept at a temperature of between 35° C. and 40° C. During the reaction, a white precipitate is formed, imidazole hydrochloride, in an amount of 17.3 g/min. Then, a batch process is applied to recover the 1,1′-carbonyldiimidazole, in an amount of 13.4 g/min.

For 8 hours of operation, around 5.4 kg of phosgene, around 13.5 kg of imidazole and around 72.9 kg of ethyl acetate solvent are consumed.

The residence time in the reactor Ts was calculated at 0.7 min.

The reaction scheme is shown below:

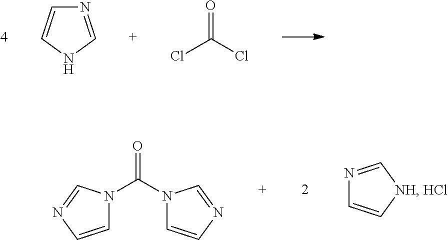

### Example 3

In the first reactor R1, a flux of 9.0 g/min (i.e. 0.127 mol/min) of Cl2 and 3.7 g/min (i.e. 0.133 mol/min) of CO (corresponding to a molar ratio CO/Cl2 of around 1.050) were reacted in a reactor according to the invention. The flux leaving the first reactor contains 12.5 g/min of phosgene and 0.2 g/l of excess CO. During the 8 hours of operation in a steady state, 6.0 kg of phosgene were thus produced from 4.3 kg of Cl2 and 1.8 kg of CO, and 0.1 kg of excess CO were recovered.

In the second reactor R2, the flux of 12.5 g/min (i.e. 0.133 mol/min) of phosgene generated in the first reactor was reacted with a liquid phase comprised of 245.1 g/min CH₂Cl₂ and 18.8 g/l (i.e. 0.257 mol/min) of dimethylformamide (DMF, CAS no. 68-12-2), corresponding to 7.1% by weight of DMF, introduced in a permanent flux into the second reactor (corresponding to a molar ratio of DMF/COCl2 of around 2). The solution introduced into the reactor had a temperature of between −5° C. and 5° C. The reaction itself took place at a temperature of around 0° C. to 5° C. The solution leaving the reactor was collected in a tank kept at a temperature of between 0° C. and 5° C. During the reaction, 16.3 g/min of (chloromethylene)dimethyliminium chloride is formed (commonly called Vilsmeier reagent, CAS no. 3724-43-4). The amount of CO2 released is 5.6 g/min. A batch process was then applied to recover the (chloromethylene)dimethyliminium chloride.

For 8 hours of operation, around 5.4 kg of phosgene, around 13.5 kg of imidazole and around 72.9 kg of ethyl acetate solvent were consumed.

The residence time in the reactor Ts was calculated at 0.7 min.

The reaction scheme is shown below:

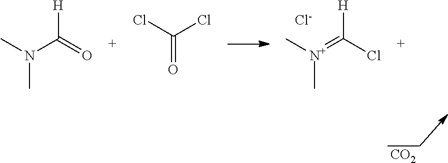

### Example 4

In the first reactor R1, a flux of 48.6 g/min (i.e. 0.686 mol/min) of Cl2 and 20.0 g/min (i.e. 0.718 mol/min) of CO (corresponding to a molar ratio CO/Cl2 of around 1.050) were reacted in a reactor according to the invention. The flux leaving the first reactor contained 67.5 g/min of phosgene and 0.2 g/l of excess CO. During the 8 hours of operation in a steady state, 32.4 kg of phosgene were thus produced from 23.2 kg of Cl2 and 9.7 kg of CO, and 0.5 kg of excess CO is recovered.

In the second reactor, this flux was reacted with a solution of 67.5 g/min (i.e. 0.674 mol/min) of cyclohexanol in toluene introduced in a permanent flux. The solution was introduced at between 10° C. to 40° C. The reaction temperature in the second reactor was maintained at between 15° C. and 45° C. The solution may contain a molar equivalent of triethylamine (TEA) enabling the HCl formed to be captured. This TEA can also be contained in the tank for receiving the flux of material at the outlet of the reactor 2. The solution containing the reaction product, namely the cyclohexyl chloroformate (CAS no. 13248-54-9) was collected at the outlet of the reactor R2 in a tank kept at around 30° C. Washings and extractions made it possible to isolate the cyclohexyl chloroformate. The latter can also be purified by distillation according to the quality required. For 8 hours of operation, 32 kg of phosgene were consumed and 12.3 kg of cyclohexyl chloroformate were produced.

The reaction scheme is shown below:

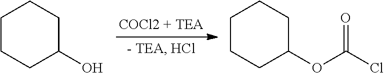 [SMILES: OC1CCCCC1 ; O=C(Cl)OC1CCCCC1]

1-19. (canceled)

20. A process for phosgenation of an organic compound into a product, the process comprising:
providing a piston reactor equipped with mechanical axial agitation means, and first reactor located upstream of the piston reactor;
generating phosgene in said first reactor from CO and Cl₂;
continuously introducing at least one liquid phase compound comprising said organic compound into said piston reactor at its upstream end, and continuously injecting the phosgene generated in said first reactor into said piston reactor at its upstream end,
subjecting said liquid phase compound, at a temperature of between −25° C. and +200° C. and under mechanical axial agitation to the influence of a phosgene pressure of between 1 and 5 bar, for a passage time of between 40 seconds and 3 minutes, so that the phosgene reacts with said liquid phase compound in order to form said product;
removing the liquid phase compound comprising said product from said piston reactor;
wherein an increase in temperature ΔT of the liquid between the inlet and the outlet of the reactor is such that the ratio ΔT/ΔT_{ad}, where ΔT_{ad} represents the adiabatic temperature increase) is between 0.02 and 0.6 when the ratio between the characteristic heat transfer time tₜₕₑᵣₘ and the characteristic material transfer time tₘₐₜ is between 1 and 50,
wherein the phosgene consumed in said piston reactor is generated continuously in the first reactor.

21. The process of claim 20, wherein the process comprises a catalytic phosgenation process.

22. The process of claim 20, wherein ΔT/ΔT_{ad} is between 0.02 and 0.2 when tₜₕₑᵣₘ/tₘₐₜ is between 1.5 and 12.

23. The process of claim 20, wherein ΔT/ΔT_{ad} is between 0.03 and 0.15 when tₜₕₑᵣₘ/tₘₐₜ is between 2 and 8.

24. The process of claim 20, wherein 3 s<tₘₐₜ<10 s.

25. The process of claim 20, wherein the heat transfer is between 300 and 700 W/m2/° C.

26. The process of claim 20, wherein the interior diameter of the piston reactor is between 40 mm and 60 mm.

27. The process of claim 20, wherein the length of the reaction chamber of the piston reactor is between 20 cm and 80 cm.

28. The process of claim 20, wherein said product is selected from the group consisting of carbonyl chlorides, chloroformates, acid chlorides, carbamates, isocyanates, carbamoyl chlorides, urea and derivatives thereof, and carbonates.

29. The process of claim 20, wherein the flow rate of the CO and Cl₂ gases in said first reactor is controlled by the phosgene consumption in said piston reactor.

30. The process of claim 20, wherein the phosgene consumption is between 1 kg/h and 50 kg/h.

31. A device for implementing a process for phosgenation of an organic compound into a product, the device comprising:
a first reactor located in a first closed compartment, said first reactor comprising a phosgene generating reactor that includes:
a first tank and an inlet for carbon monoxide gas with a second flow regulator;
a second tank and an inlet for chlorine gas with a second flow regulator thereof;
a reaction chamber having a fixed bed catalyst enabling a carbon monoxide-chlorine gas mixture to be converted into phosgene; and
a second reactor located in a second closed compartment and downstream of the first reactor, said second reactor comprising a tubular phosgenation reactor that includes:
at an upstream end thereof, an inlet for a gaseous reaction product coming from said first reactor;
at the upstream end, an inlet for a liquid phase compound comprising said organic compound,
at a downstream end thereof, an outlet for said liquid phase compound,
wherein:
said first closed compartment and said second closed compartment are separated by a wall;
said first reactor and said second reactor are connected to one another by a tube for transfer of the reaction product comprising the phosgene generated in said first reactor to said second reactor, with said tube passing through said wall between said first compartment and said second compartment;
said second reactor includes mechanical axial agitation means; and
said second reactor operates at a pressure of between 1 and 5 bar.

32. The device of claim 31, wherein said first compartment and said second compartment are under a negative pressure, with their respective gas content being continuously suctioned and sent through a wet scrubber configured to destroy the phosgene and the chlorine gas that said gas content contains.

33. The device of claim 31, wherein the interior diameter of the second reactor is between 40 mm and 60 mm.

34. The device of claim 31, wherein the length of the reaction chamber of the second reactor R2 is between 20 cm and 80 cm.

35. The device of claim 31, wherein said transfer tube includes means for cooling the phosgene.

36. Using a device for the phosgenation of an organic compound into a product, the device comprising:
a first reactor located in a first closed compartment, said first reactor comprising a phosgene generating reactor that includes:
a first tank and an inlet for carbon monoxide gas with a second flow regulator;
a second tank and an inlet for chlorine gas with a second flow regulator thereof;
a reaction chamber having a fixed bed catalyst enabling a carbon monoxide-chlorine gas mixture to be converted into phosgene; and
a second reactor located in a second closed compartment and downstream of the first reactor, said second reactor comprising a tubular phosgenation reactor that includes:
at an upstream end thereof, an inlet for a gaseous reaction product coming from said first reactor;
at the upstream end, an inlet for a liquid phase compound comprising said organic compound,
at a downstream end thereof, an outlet for said liquid phase compound,
wherein:
said first closed compartment and said second closed compartment are separated by a wall;
said first reactor and said second reactor are connected to one another by a tube for transfer of the reaction product comprising the phosgene generated in said first reactor to said second reactor, with said tube passing through said wall between said first compartment and said second compartment;
said second reactor includes mechanical axial agitation means; and
said second reactor operates at a pressure of between 1 and 5 bar.

37. The use of claim 36, wherein the product is selected from the group formed by carbonyl chlorides, chloroformates, acid chlorides, carbamates, isocyanates, carbamoyl chlorides, urea and derivatives thereof, and carbonates.
